(19) United States
(12) Patent Application Publication
(10) Pub. No.: US 2006/0224125 A1
(43) Pub. Date: Oct. 5, 2006

(54) Multiport syringe

(75) Inventors: Philip J. Simpson, Escondido, CA (US); David G. Matsuura, Encinitas, CA (US)

Correspondence Address: BUCHANAN, INGERSOLL & ROONEY PC, POST OFFICE BOX 1404, ALEXANDRIA, VA, 22313-1404, US

(73) Assignee: VASOGEN IRELAND LIMITED, Shannon (IE)

(21) Appl. No.: 11/373,211

(22) Filed: Mar. 13, 2006

Related U.S. Application Data
(60) Provisional application No. 60/662,214, filed on Mar. 15, 2005.
(60) Provisional application No. 60/663,030, filed on Mar. 16, 2005.

(51) Int. Cl. A61M 5/315; A61M 5/00

(52) U.S. Cl. 604/236; 604/191

(57) ABSTRACT

A multi-port syringe with a fluid chamber in communication with an inlet valve assembly and an outlet valve assembly disposed intermediate the fluid chamber and the inlet valve assembly, the inlet valve assembly has an inlet valve member operable in an open position and a closed position, the inlet valve member being biased in a closed position by a resilient member. The outlet valve assembly has an outlet valve member operable between a closed position and an open position, and the outlet valve member is maintained in a closed position by a sealing member, and includes anchoring member engaging the outlet valve assembly. With the outlet valve member in the closed position, the inlet valve member is forced open by compressing the resilient member, thus allowing fluid into the chamber, and with the fluid chamber primed and the inlet valve member in a closed position, the outlet valve member is forced open by disengaging the anchoring member and defeating the sealing member to allow fluid discharge from the chamber.

### CROSS-REFERENCE TO RELATED APPLICATIONS

This application claims the benefit of priority to U.S. Provisional Application Ser. No. 60/662,214, filed Mar. 15, 2005, and 60/663,030, filed Mar. 16, 2005.

### BACKGROUND OF THE INVENTION

1. Field of the Invention

The present invention relates to material dispensers, more particularly it relates to multi-port syringes.

2. Description of the Prior Art

Most of all pharmaceuticals used in hospitals are now administered by injection, either directly or through injection sites. Similarly, blood or other fluid sampling using hypodermic syringes and evacuated chambers has become common place. Accordingly, health-care workers are continuously exposed to needle sticks and to patient bodily fluids. Statistics show that most needle sticks occur during recapping of used needles, picking up and carrying the needle, placing needles in a receptacle, or when needles are inadvertently left at a patient's bedside. Despite safety guidelines and employee education, there is little evidence that their incidence is abating.

Over the years a wide variety of devices and methods have been developed to protect the health care practitioner as well as patients from needle sticks. An example of such a device is a valve adapter device that is releasably attached to a syringe and adapted to fit to medicinal bottles. The adapter acts as a channel for fluid transfer from medicinal containers, IV ports and other administrative structures for medical purposes.

Although the adapter devices may help diminish the chance of needle-sticks, they do suffer a number of disadvantages. Also, since an adapter device is an additional part to a syringe, this results in higher costs for the health facility, and also higher patient-care costs. There is also a greater chance that an adapter device is likely to be used more than once, thus there is a risk of cross-contamination.

It is thus one of the objects of this invention to mitigate or obviate at least one of the aforementioned disadvantages.

### SUMMARY OF THE INVENTION

In one of its aspects, the present invention provides a syringe having:

a fluid chamber;

an inlet valve assembly in communication with the fluid chamber; and

an outlet valve assembly disposed intermediate the fluid chamber and the inlet valve assembly;

the inlet valve assembly including:
an inlet valve member operable in an open position and a closed position;
a resilient member biasing the inlet valve member to its closed position;

the outlet valve assembly including:
an outlet valve member operable between a closed position and an open position; and
a sealing member; and
an anchoring member engaging the outlet valve assembly to maintain the outlet valve member in a closed position;
wherein;

with the outlet valve member in the closed position, the inlet valve member is placed into the open position upon compression of the resilient member to allow fluid flow into the chamber;

and the outlet valve member is operable by disengaging the anchoring means and defeating the sealing means when the fluid chamber is primed, while the inlet valve member is in a closed position;

thereby to allow discharge from the chamber.

In another of its aspects, the present invention provides a multi-port syringe having:

a housing with a proximal end and a distal end;

an inlet valve assembly adjacent to the proximal end;

an outlet valve assembly adjacent to the proximal end;

the inlet valve assembly and the outlet valve assembly being angled with respect to one another;

the distal end of the housing having a chamber therein;

the inlet valve assembly having:

an inlet opening, a first tubular portion leading to which the inlet opening leads, and a second tubular portion;

an inlet valve member received by the first tubular portion and the second tubular portion, to operate the inlet opening between an open position and a closed position;

the inlet valve member being dimensioned to occupy a substantial volume of the first tubular portion and the second tubular portion while defining a first cavity between the inlet valve member and the walls of the first tubular portion and defining a second cavity between the inlet valve member and the walls of the second tubular portion; and

a resilient member for biasing the inlet valve member in a closed position;

the outlet valve assembly having:
a third tubular portion having an outlet opening and an activation opening, and disposed intermediate the second tubular portion and the chamber in a cruciform arrangement, and in communication therewith;
the third tubular portion for receiving an outlet valve member to operate the outlet opening between a closed position and an open position;
the outlet valve member being dimensioned to occupy a substantial volume of the third tubular portion while defining a third cavity between the valve means and the walls of the third tubular portion;
the outlet valve member having:
   a first annular ring engaging the walls of the third tubular portion to seal the third tubular portion adjacent to the activation opening; and
   a second annular ring engaging the walls of the third tubular portion to seal the third tubular portion adjacent to the outlet opening;
   a plurality of lugs for engaging an annular recess in the walls of the third tubular portion to anchor the outlet valve member to maintain the outlet opening in the closed position;

wherein with the outlet opening in the closed position, the inlet valve member is driven towards the second tubular portion and the resilient member is compressed to open the inlet opening to allow fluid to flow via the first cavity and the second cavity into the chamber; and

wherein with the inlet opening in a closed position, the lugs are forced out of the annular recess such that a second annular ring completely disengages the walls of the second tubular portion to unseal the outlet opening and allow discharge from the chamber via the third cavity.

Advantageously, a variety of devices can be interchangeably attached to the syringe, such as a butterfly needle, a sodium citrate bag, in order to draw one or more fluids into the chamber, or express fluid from the chamber. Additionally, the outlet valve member is only operable from a closed position to an open position, thus making the syringe a single-use device, which diminishes cross-contamination risks common with multiple-use devices. Also, by having sharps-free valves, the risk of needle-stick injuries is reduced.

In one example, the syringe is used in a system for the collection, treatment and delivery of an autologous blood sample. The syringe is used to collect an untreated blood sample from an originating patient. Following blood sample collection, the first syringe is connected to a blood treatment chamber which is then delivered to a blood treatment unit, in which the blood sample is subjected to one or more stressors as, for example, described in International Publication No. WO0119318A1 entitled “APPARATUS AND PROCESS FOR CONDITIONING MAMMALIAN BLOOD” (the entire contents of which are incorporated herein by reference).

### BRIEF DESCRIPTION OF THE DRAWINGS

These and other features of the preferred embodiments of the invention will become more apparent in the following detailed description in which reference is made to the appended drawings wherein:

FIG. 1 is a plan view of a multi-port syringe;

FIG. 2 is a sectional view of part of the multi-port syringe of FIG. 1 taken along line 2-2′ of FIG. 1;

FIG. 3 is an isometric view of the valve plunger of the inlet valve assembly of the syringe of FIG. 1;

FIG. 4 is a portion of the sectional view of the inlet valve assembly taken along line 2-2′ of FIG. 1;

FIG. 5 is an exploded view of the inlet valve assembly;

FIG. 6 an isometric view of the shell body of the inlet valve assembly;

FIG. 7 is a sectional view of the outlet valve assembly of the syringe of FIG. 1 taken along line 2-2′ of FIG. 1;

FIG. 8 is an exploded view of the outlet valve assembly;

FIG. 9 is an isometric view of the valve plunger of the outlet valve assembly;

FIG. 10a is a side view of the syringe of FIG. 1 in a mating relationship with a

sodium citrate bag;

FIG. 10b is a plan view of the syringe of FIG. 1 in a mating relationship with a

sodium citrate bag;

FIG. 11 is a longitudinal sectional detailed view the syringe of FIG. 1 in a

mating relationship with a sodium citrate bag taken along the line 10-10′ of FIG. 10b;

FIG. 12 is a sectional view of FIG. 10b, showing a male coupler in a mating

relationship with a female coupler, taken along the line 10-10′;

FIG. 13 is a sectional view of the valve assembly of FIG. 11 in a closed state;

FIG. 14 is a sectional view of the valve assembly FIG. 11 in an open state;

FIG. 15 is a plan view of a multi-port syringe of a second embodiment; and

FIG. 16 is a sectional view of the multi-port syringe of FIG. 1 taken along line 15-15′ of FIG. 15.

### DESCRIPTION OF THE PREFERRED EMBODIMENTS

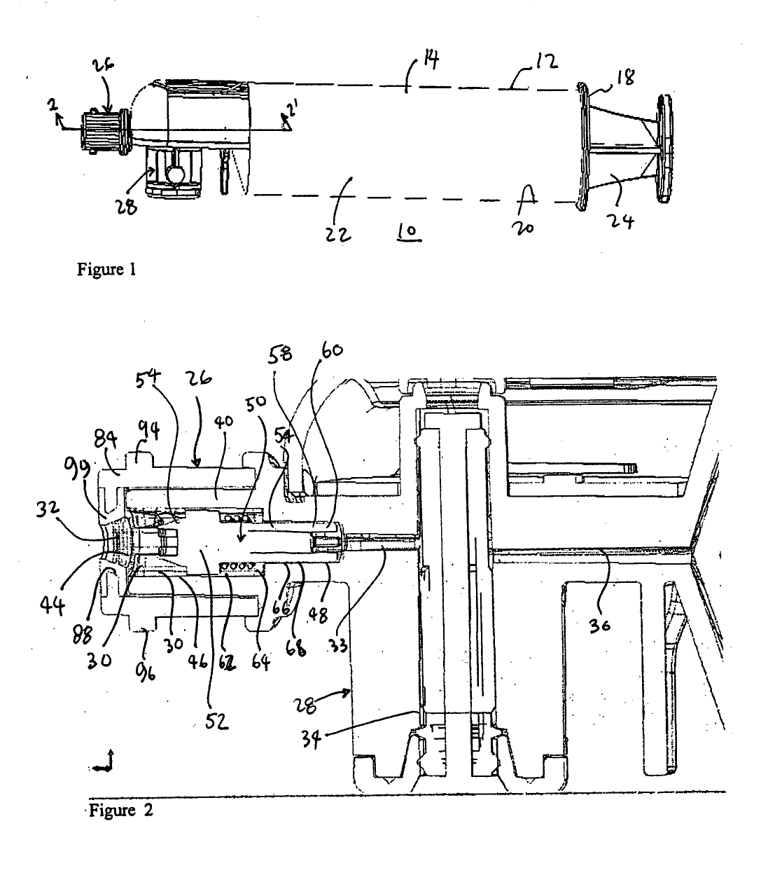

Referring FIG. 1, there is shown a fluid dispenser, such as a multi-port syringe 10, in a preferred embodiment.

The syringe 10 includes a body 12 having a syringe barrel 14 with an open proximal end 16 at which are disposed an inlet valve assembly 26 and an outlet valve assembly 28, a distal end 18 and a cylindrical wall 20 therebetween to define a fluid receiving chamber 22. A plunger 24 is slidably disposed at the distal end 18 of the syringe 10 and is in fluid-tight engagement with the cylindrical wall 20. The plunger 24 serves to draw fluid into the chamber 22 or urge fluid from the chamber 22, as will be explained further herein.

Referring to FIG. 2, there is illustrated the inlet valve assembly 26 for controlling the ingress of fluid into the chamber 22, and the outlet valve assembly 28 for controlling the egress of fluid from the chamber 22. The outlet valve assembly 28 is disposed at an angle with the inlet valve assembly 26 and intermediate the chamber 22 and the inlet valve assembly 26. As shown, the inlet valve assembly 26 and the outlet valve assembly 28 are perpendicular to each other. A fluid communication channel 30 leads from the proximal end 16 to the fluid receiving chamber 22. The fluid communication channel 30 includes a bore 32 extending from the proximal end 16 and leading to a first lumen 33, an outlet tubular portion 34 within the outlet valve assembly 28, and a second lumen 36 extending from the outlet tubular portion 34 into the chamber 22. The outlet tubular portion 34 is thus perpendicular to the first lumen 33 and the second lumen 36, and is dimensioned to accommodate the outlet valve assembly 28.

The inlet valve assembly 26 will now be described in greater detail, with reference to FIG. 2. The inlet valve assembly 26 is a hollowed out cylindrical shell 40 containing the bore 32, and having an opening 44. The bore 32 includes tubular portions 46, 48 in communication with the first lumen 33. The diameter of the tubular portion 46 is greater compared to that of the tubular portion 48, and contained within the tubular portions 46 and 48 is a valve plunger 50 which operates to open and close the opening 44. The valve plunger 50 is a cylindrical rod comprising a head 52 with a tapered head portion 54, a stem 56 and a tail portion 58 with detents 60, as shown in FIG. 4. The stem 56 is of reduced diameter compared to the valve head 52, to accommodate and complement the tubular portions 46 and 48, respectively. As such, there is defined a cavity 62 between the head 52 and the wall 64 of the tubular portion 46, and correspondingly a cavity 66 is defined between the stem 56 and the wall 68 of the tubular portion 48. The cavities 62 and 66 permit flow of fluid though the communication channel 30.

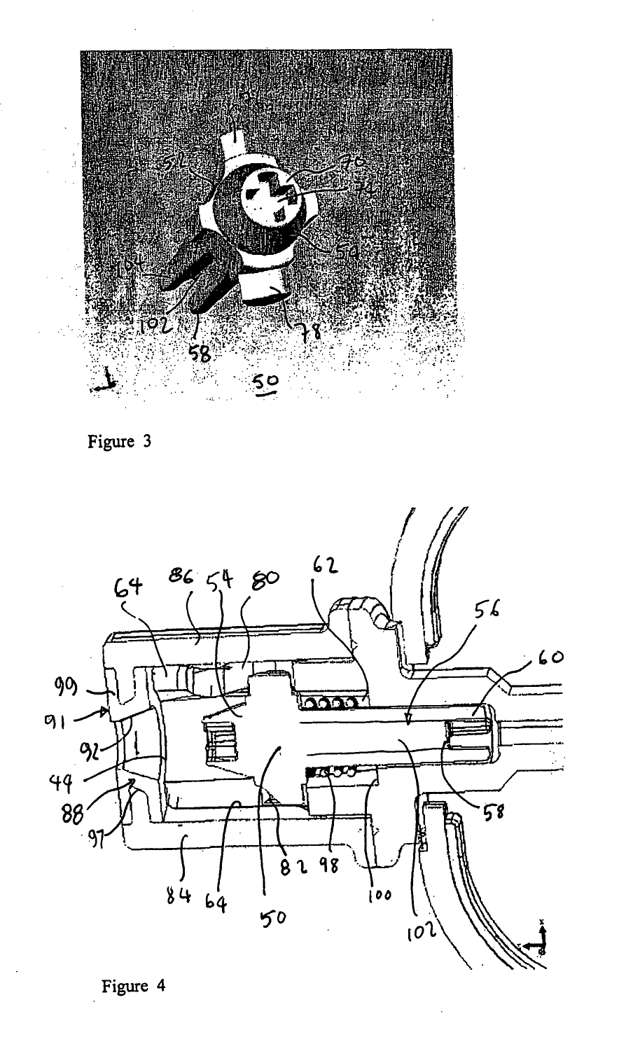

As shown in FIGS. 3 to 6, the tapered head portion 54 of the valve plunger includes a tip 70 having an axially presented surface 72 facing the opening 44. The axially presented surface 72 includes a cruciform recess 74 engageable by a complementary male cruciform head 202 to operate the inlet valve assembly 26, as will be explained later in connection with FIG. 12. The valve head 52 includes two opposed bayonet pins 76 and 78 which engage slots 80, 82 in the wall 64 of tubular portion 46. The slots 80, 82 thus provide guide means for the valve plunger 50 along the tubular portions 46 and 48, to facilitate opening and closing the opening 44. The slots 80, 82 include axially presented openings 81, 83, end portions 85, 87 remote from the openings 81, 83, locking ramp sections 89, 91 and locking portions 93, 95.

Disposed around the shell 40 is a male coupler 84 with a rotatable coupling sleeve 86 with a sleeve opening 88 contiguous with the opening 44, as shown in FIG. 4. The coupling sleeve 86 includes a recessed, annular, bevelled flange 97 with a cap 99 which maintains the valve plunger 50 within the inlet valve assembly 26. The cap 99 is a comolded elastomer or rubber which envelops the flange 97. The cap 99 thus includes an annular face 91, and a tapered bore 92 to receive the tapered head portion 54, such that at least one portion of the valve head 56 cooperates or engages the tapered bore 92 to seal the opening 44. The rotatable coupling sleeve 86 also includes a pair of diametrically opposed bayonet pins 94, 96 to engage slots 219,220 on complementary female coupler 176 of another device 177, as is shown in FIG. 12 and described below.

A resilient member 98 such as a helical spring, a rubber or a polymer elastomer, biases the valve plunger 50 into a closed position. As shown, the resilient member 98 is a spring which biases the tapered head portion 54 into the tapered bore 92 to seal the opening 44, thus maintaining the valve plunger 50 in a closed position. The spring 98 loosely engages a portion of stem 56 and acts between the bayonet pins 94, 96, shown in FIG. 2, and an end portion 100 of the tubular portion 46.

The stem 56 is dimensioned such that a significant part thereof resides in the tubular portion 46, regardless of whether the valve plunger 50 is in a closed position or open position, and so the tail portion 58 is always within the tubular portion 48. The stem 56 includes an elongate rod 102 extending from the valve head 54 to the tail portion 58. Also, extending from the valve head 54, and terminating beyond the tail portion 58, are four generally parallelepiped ribs 104 positioned generally perpendicular to each other on the elongate rod 102, or made integrally with the elongate rod 102. The parallelepiped ribs 104 extend beyond the elongate rod 102 to form detents 60. The ribs 104 and the detents 60 are so dimensioned to occupy a large volume of the tubular portions 46 and 48, while allowing sufficient clearance between the walls and the valve head, that is, cavities 62 and 66, for fluid to flow without substantial impedance. The cruciform arrangement of the ribs 104 and detents 60 limits the tubular portions 46 and 48 within the cavities 62 and 66 where fluid can accumulate, and may consequently render the fluid unusable, in the case of blood.

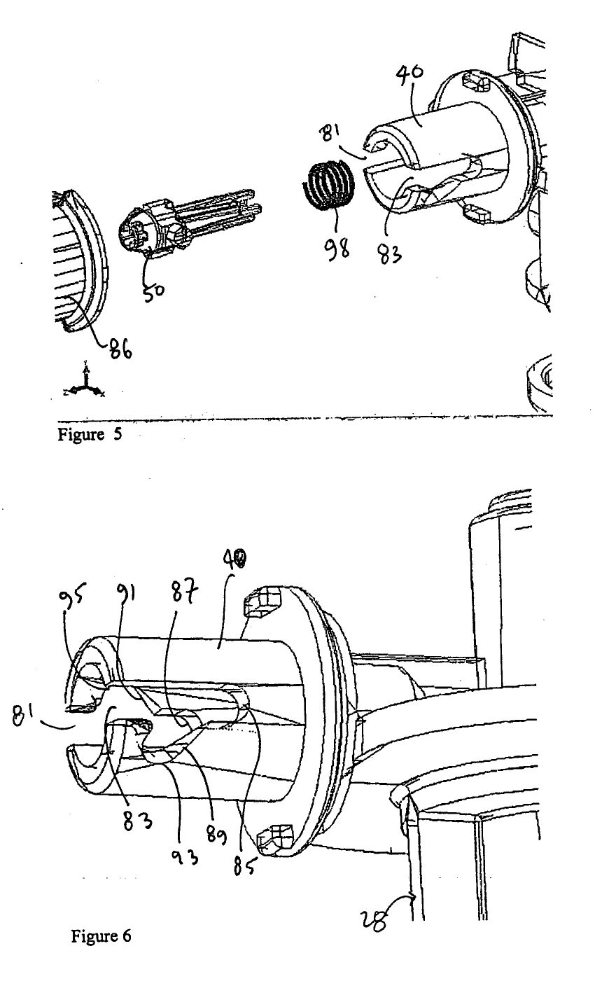

As stated above, two diametrically opposed slots 80, 82 are formed in the wall 64 of tubular portion 46. As shown in FIGS. 5 to 6, the slots 80, 82 extend from the opening 44 and terminate at endpoints 85, 87, close to the distal end of the tubular portion 46. The locking ramp sections 89, 91 serve to draw the bayonet pins 76, 78 towards the locking portions 93, 95, under the force of the spring 98.

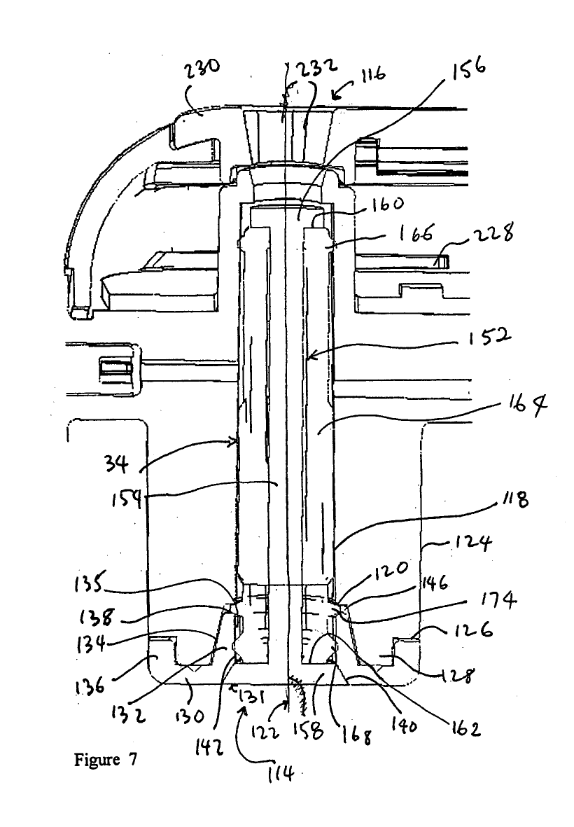

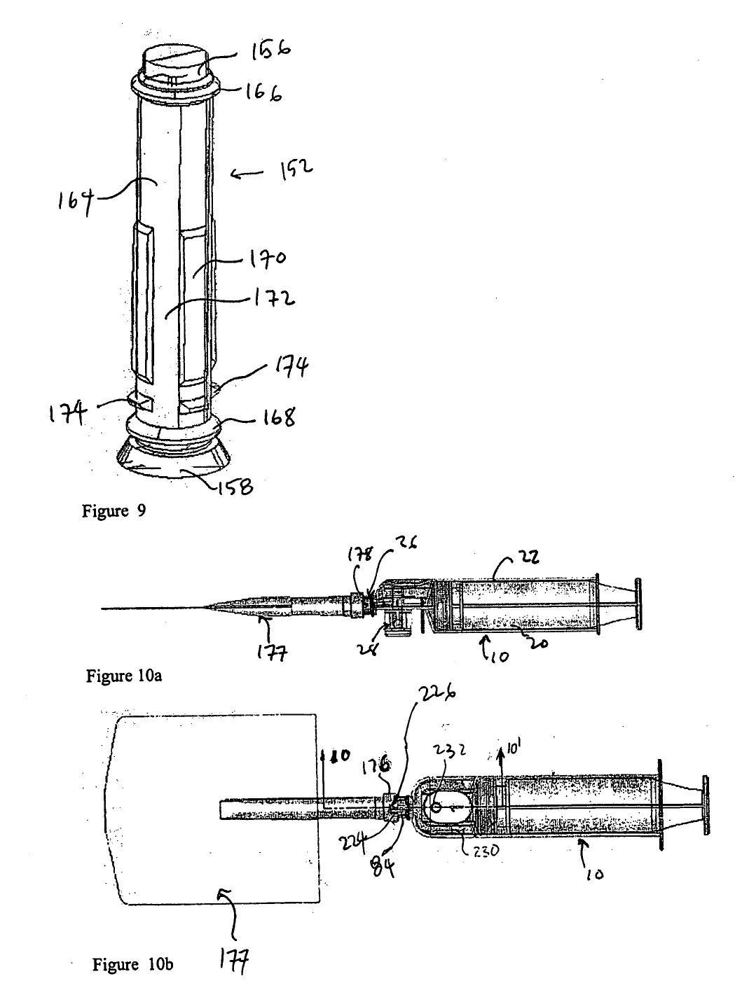

The outlet valve assembly 28 will now be described in greater detail, with reference to FIGS. 7 to 9. The outlet valve assembly 28 includes an outlet tubular portion 34 with an open outlet end 114 and an open activation end 116. Near the outlet end 114, an inner wall 118 of the outlet tubular portion 34 has a peripheral margin 120 sloped away from the longitudinal centerline 122 of the outlet tubular portion 34 and an outer wall 124 has an annular recess 126 which defines an outlet rim 128 with an annular configuration. An end cap 130 is secured to the outlet end 114, and includes an annular housing with a bore 131 and a skirt 132 having an outer skirt wall 134 engaging the inner wall 118 of the outlet tubular portion 34, an inner skirt wall 135. A flange 136 engages the outlet rim 128 and the annular recess 126 of the outlet tubular portion 34. A skirt edge 138 is defined at the apex of the inner skirt wall 135 and the outer skirt wall 134, such that an annular recess 146 is defined between the skirt edge 138 and the peripheral margin 120. The bore 131 includes a frustoconical bore portion 140 adjacent to the outlet end 114, and a cylindrical bore portion 142 having about the same diameter as the outlet tubular portion 34.

The outlet tubular portion 34 houses an outlet valve plunger 152, so configured to prevent fluid from escaping from the outlet tubular portion 34, and to allow the outlet valve plunger 152 to be urged from a sealing position, via the activation end 116, to an open position wherein the fluid flows from the outlet tubular portion 32 via outlet end 114.

The outlet valve 152 includes a cylindrical stem 154 with a valve head 156 adjacent to the activation end 116, and a frustoconical valve base 158 adjacent to the outlet end 114. The stem 154 is of slightly reduced diameter than the valve head 156, such that the valve head 156 and stem 154 define a lip 160. Correspondingly, another lip 162 is defined between the stem 154 and the valve base 158. Provided between the lip 160 and the lip 162 is a sleeve 164 which wholly envelops the stem 154, and serves to anchor the outlet valve plunger 152 within the outlet tubular portion 34, while permitting fluid flow though the outlet tubular portion 32 during fluid withdrawal or fluid expression from the syringe 10.

The sleeve 164 comprises a resilient material, such as rubber or a polymeric elastomer. Adjacent to the lip 160 is a first annular ring 166 off the sleeve 164 which engages a sufficient circumferential area of the wall 118 of the outlet tubular portion 34 to provide a seal to the fluid. Correspondingly, adjacent to the lip 162 is a second annular ring 168 off the sleeve 164 which engages a sufficient circumferential area of the wall 118 of the outlet tubular portion 34 to provide a seal to the fluid. The first annular ring 166 and the second annular ring 168 are made integral with the sleeve 164 and provide an interference seal with walls 118 to contain the fluid within the outlet tubular portion 34. The sleeve 164 also includes a plurality of longitudinally extending ribs 170 which slidably engage the wall 118. The ribs 170 are located between the first annular ring 166 and the second annular ring 168, and are equally spaced apart around the sleeve 164 to define a plurality of cavities 172 between same. The outlet valve plunger 152 is dimensioned to occupy a large volume of the outlet tubular portion 34 while allowing sufficient clearance between the walls 118 for fluid to flow without substantial impedance via the cavities 172. The ribs 170 limit substantially diminish the chances of fluid accumulating within the outlet tubular portion 34 by limiting the blood to the cavities 172, a particular advantage when it is preferred the apparatus is used for the handling of biological fluid such as blood. Between the ribs 170 and the second annular ring 168 are lugs 174, formed integral with the sleeve 164, located along the same longitudinal axis as the ribs 170. The lugs 174 serve to engage the annular recess 146 and thus anchor the valve plunger 152 in place within the outlet tubular portion 34, until a force to open the valve 152 is applied to the valve head 156 to force the lugs 174 out of the annular recess 146. Once the lugs 174 have been forced out of the annular recess 146 and the second annular ring 168 is no longer in contact with cap 130, the outlet valve plunger 152 can not be forced back into the outlet tubular portion 34.

The operation of the syringe 10 will now be described, specifically the process of drawing one or more fluids into the chamber 22 via different devices that can be coupled to the to the inlet valve assembly 26 of the syringe 10. As an example, when the syringe 10 is used to draw blood from a patient, a blood anti-coagulant, such as sodium citrate solution, is also drawn in to the chamber 22 to prevent clotting of the blood.

Starting in the rest or closed position, the valve plunger 50 is biased against the tapered bore 92 by the spring 98 and the pins 76, 78 are in the locking portions 93, 95 of slots 219,220. Correspondingly, the lugs 174 of the outlet valve head plunger 152 engage the annular recess 146 to anchor the outlet valve plunger 152 within the outlet tubular portion 34 in the rest or closed position. The first annular ring 166 and the second annular ring 168 seal the activation end 116 and the outlet end 114 respectively.

FIGS. 10a and 10b shows a male coupler 84 of the syringe 10 in a mating arrangement with a female coupler 176 of a sodium citrate bag 177. The female coupler 176 is inserted into a tubing of a sodium citrate bag 177. The female coupler 176 comprises a complementary valve assembly 178, which when mated with the male coupler 84 causes the valve assembly 178 to open and allow passage of fluid therethrough.

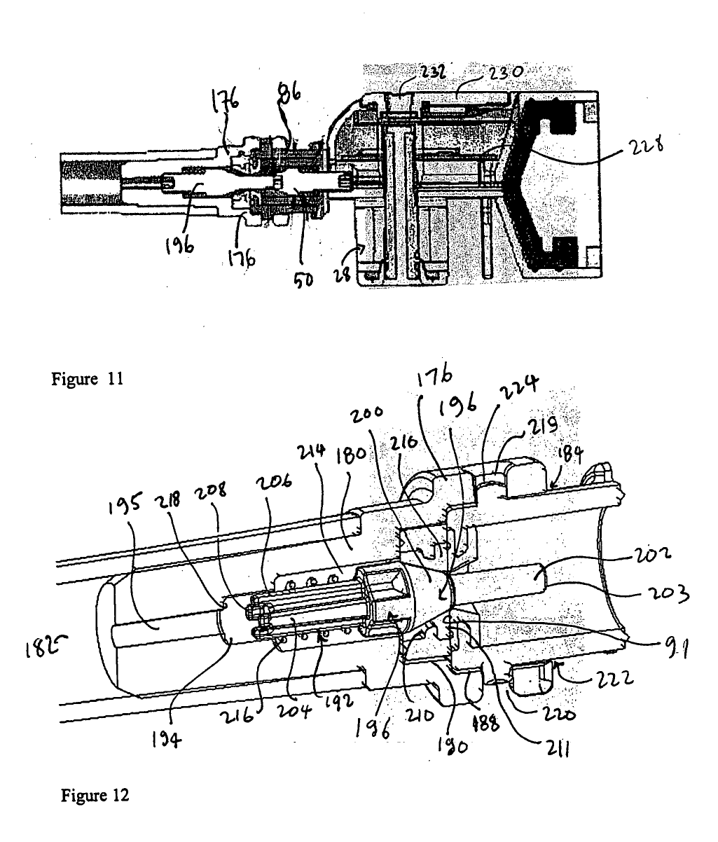

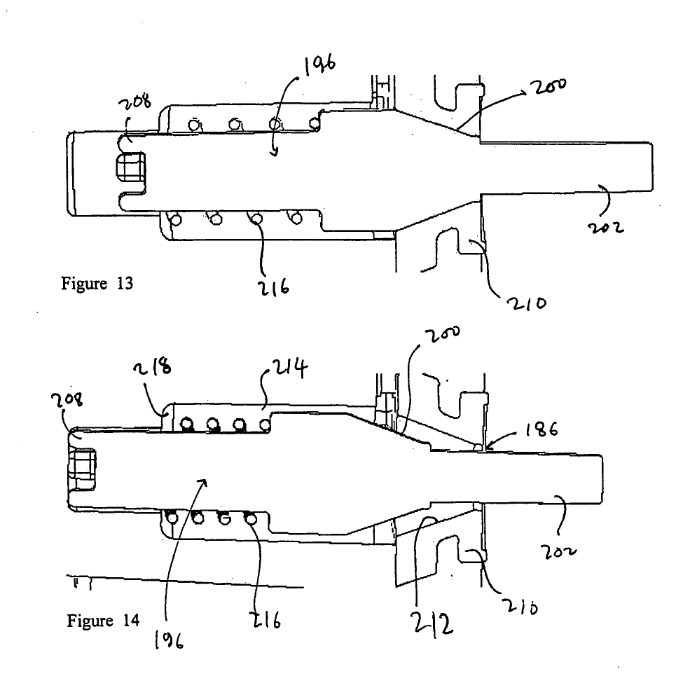

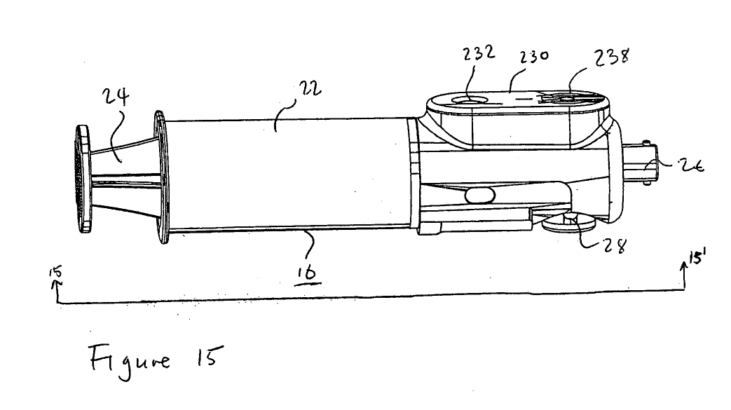

In more detail, as shown in FIGS. 11-14, the female coupler 176 includes a hollowed out shell 180 with two ends 182, 184 having a recessed opening 186 at one end 184. A bore 186 with tubular portions 188, 190, 192, 194 and 195 of reduced diameters extends from one end 182 to the other end 184. The valve assembly 178 includes a valve plunger 196 that resides in tubular portions 188, 190, 192 and 195. The valve plunger 196 is a cylindrical rod comprising a valve head 198 with a tapered head portion 200 which merges into a nipple 202 with lugs 203, a stem 204 and a tail 206 with detents 208. The plunger 196 is maintained within the shell 180 by a cap 210 which is a comolded elastomer, with an annular face 211, having a tapered bore 212 to receive the tapered head portion 200. The valve plunger 196 is dimensioned to accommodate as much volume as possible of the tubular portions 192, 194, while allowing fluid flow therethrough. The stem 204 is of reduced diameter compared to the valve head 198, such that there exists a cavity 214 between the stem 204 surface and the tubular portion 192 inner wall. The cavity 214 includes a resilient member 216, such as a helical spring extending between the valve head 198 and an end portion 218 of the tubular portion 192. Preferably, the resilient member 216 is a spring which biases the tapered head portion 200 into the tapered bore 212 to seal the opening 186, thus maintaining the valve plunger 196 in a closed position. Applying an axial force on the nipple 202 in the direction as shown, compresses the spring 216 and urges the tapered head portion 200 away from the tapered bore 212 to break the seal at opening 186 thus placing the valve assembly 178 into an open position.

The shell 180 includes two diametrically opposed slots 219, 220 extending from the opening edge 222, which receive the bayonet pins 94, 96 on the male coupler 84. The slots 219, 220 are generally L-shaped with a guide portion 224 and a locking portion 226.

The male coupler 84 and the female coupler 176 are brought together and the bayonet pins 94, 96 are aligned with slots 219,220. The pins 94, 96 engage the guide portion 224 and an axial force applied to either coupler 84 or 176 urges the pins 94, 96 along the guide portion 224 and a torque on rotatable sleeve 164 of syringe 10 urges the pins 94, 96 from the guide portion 224 into the locking portion 226. In this position, the annular faces 91, 211 of caps 90, 210 are forced against each other as the caps 90, 210 compress against each other to form a tight seal and thus establish a channel between the two couplers 84,176. At the same time, the nipple head lugs 203 engage the cruciform recess 74 of the valve head 52 and the axial force urges the bayonet pins 94, 96 out of the locking portions 93, 95 along the ramp portion 89, 91 to end portion 85, 87 of the slots 80, 82. The axial force is thus sufficient to overcome the spring force and thus causes the spring 98 to compress. As the pins 76, 78 are urged towards the end portions 85, 87; the tapered valve head portion 54 is urged away from the frustoconical bore 92 to unseal the opening 44.

Correspondingly, in valve assembly 178, the tapered valve head portion 200 is urged away from the tapered bore 212 to open the valve assembly 178. This arrangement persists as long as the couplers 84,176 are in a mating relationship with the pins 94, 96 of coupler 84 are locked in the locking portions 226. Withdrawing the plunger 24 away from lumen 36, and along the barrel walls 20, creates a partial vacuum in the chamber 22 and thus causes fluid to flow from the sodium citrate bag 177, via the open valve assembly 178, the channel defined between the annular faces 91,211, the open valve assembly 50, the channel 30 into the chamber 22.

With the syringe 10 primed, the sodium citrate bag 177 is detached from the syringe 10, or vice versa. This is achieved by applying an opposite torque on the rotatable coupling sleeve 86 of valve assembly 28 to force the pins 94, 96 from the locking portions 226 to disengage the pins 94, 96 from the guide portions 224. In response to these actions, the spring 98 of valve assembly 28 decompresses and its spring force urges the pins 74, 76 along locking ramp 89, 91 to the end portions 93, 95 and the tapered valve head 54 is forced into the frustoconical bore 92, thus returning the valve assembly 28 into the rest or closed position. Correspondingly, in valve assembly 178, uncoupling the couplers 84,176, causes the spring 216 to decompress and force the tapered portion 200 into the tapered bore 212, to seal the opening 186, and thus returning the valve assembly 178 into the rest or closed position.

With the sodium citrate in the barrel, blood is drawn into the syringe by attaching the syringe 10 to a butterfly needle (not shown) with a valve assembly similar complementary valve assembly 178. By following similar actions as described above, blood is drawn into the syringe 10.

With the valve assembly 28 closed, the blood and sodium citrate solution can now be discharged from the outlet valve assembly 28. The outlet valve assembly 28 is coupled to an inlet valve assembly of a receptacle (not shown), such as a blood treatment chamber. The outlet valve assembly 28 is preferably coupled to the chamber by bayonet pins 228 which engage corresponding slots (not shown) of the inlet valve assembly, in a manner as described above. An axial force is then applied to the valve head 156, via the activation end 116, to urge the lugs 174 out of the annular recess 146, and thus the valve base 158 is forced away from the cap 130. The force is sufficient to clear the second annular ring 168 of the inner skirt wall 135 to unseal the outlet tubular portion 34. However, the first annular ring 168 is maintained within the outlet tubular portion 34 to maintain the seal at the activation end 1 16. The fluid then flows from the chamber 22, via the outlet tubular portion 34 and through the outlet end 114 and into the receptacle chamber. Advantageously, the outlet valve plunger 152 is only operable from a closed position to an open position, thus making the syringe 10 is a single use device which prevents contamination through multiple use.

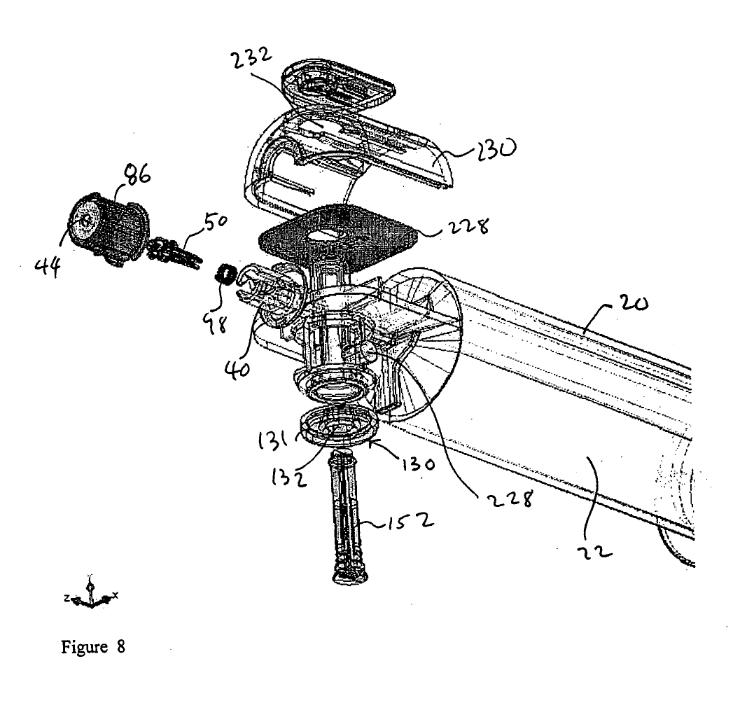

FIG. 8 shows an exploded view of the inlet valve assembly 26 and the outlet valve assembly 28. Adjacent to the activation end 116 and surrounding a portion of the outlet valve assembly 28 is a printed circuit board (PCB) 228 having circuitry for transmitting and receiving data related to the syringe or a patient, such as identification data, SKU, serial no., manufacturing date, expiry date, fluid data, patient data, health facility data, health practitioner data, medication data, and so forth. The circuitry includes, but is not limited to, a transmitter, a receiver, a processor, a memory, an antenna and a power source. As an example, the circuitry may be implemented as a radio identification (RFID) integrated circuit coupled to an antenna. The (RFID) integrated circuit may be passive, semi-active or active. The activation end 116 also includes a cover 230 having a bore 232 contiguous with the opening of the activation end 116. The bore 232 helps to prevent accidental force being applied to the valve head 156, when the syringe 10 is not in use.

In a second embodiment, the outlet valve plunger 152 is forced open by an axial force applied to the valve base 158. The outlet valve plunger 152 is biased to a closed position against a valve seat 158 by a resilient means, such as a spring or an elastomer. The outlet valve plunger 152 is aligned for abutment with a valve actuating element which is positioned in an inlet to the chamber. The valve actuating element is operable to displace the outlet valve plunger 152 from its closed position against the valve seat 158 to break the seal and allow fluid flow into the chamber.

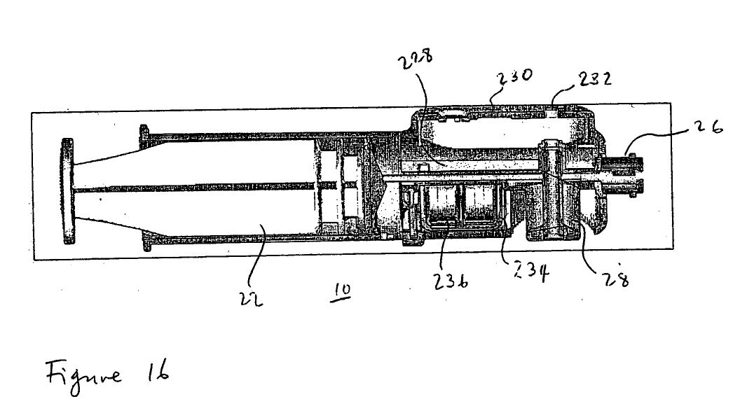

In a third embodiment, as shown in FIG. 15 and 16, the syringe 10 includes a compartment 234 for housing a power supply unit 236 to provide electrical power to a PCB 228. The power supply unit 236 typically comprises of one or more batteries, and a power circuit resident on the PCB 228 for regulating the power to the PCB and other components such as an LED 238, a display, or a piezo-electric speaker for providing output to a user. The batteries 236 may be removed after the single use of the syringe 10, in order to enable use in another syringe or allow for proper recycling in compliance with current environmental regulations.

Even though the description above is in large part focused on the use of syringe 10 in the treatment of autologous blood samples, it will be understood that the syringe 10, may be used with samples other than blood samples, such as bone marrow or, lymphatic fluids, semen, ova-fluid mixtures, other bodily fluids or other medical fluids which may or may not be “autologous”, for example fluid mixtures perhaps containing a patient's desired solid sample such as from organs, body cells and cell tissue, skin cells and skin samples, spinal cords.

Although the invention has been described with reference to certain specific embodiments, various modifications thereof will be apparent to those skilled in the art without departing from the spirit and scope of the invention as outlined in the claims appended hereto.

The embodiments of the invention in which an exclusive: Property or Privilege is claimed are defined as follows:

1. A syringe having:
a fluid chamber;
an inlet valve assembly in communication with the fluid chamber; and
an outlet valve assembly disposed intermediate the fluid chamber and the inlet valve assembly;
the inlet valve assembly including:
an inlet valve member operable in an open position and a closed position;
a resilient member biasing the inlet valve member to its closed position;
the outlet valve assembly including:
an outlet valve member operable between a closed position and an open position; and
a sealing member; and
an anchoring member engaging the outlet valve assembly to maintain the outlet valve member in a closed position;
wherein;
with the outlet valve member in the closed position, the inlet valve member is placed into the open position upon compression of the resilient member to allow fluid flow into the chamber;
and the outlet valve member is operable by disengaging the anchoring means and defeating the sealing means when the fluid chamber is primed, while the inlet valve member is in a closed position;
thereby to allow discharge from the chamber.

2. The syringe of claim 1 wherein the inlet valve member is dimensioned to occupy a substantial volume of the first tubular portion and the second tubular portion while defining a first cavity between the female inlet valve member and the walls of the first tubular portion.

3. The syringe of claim 2 wherein the inlet valve member and the walls of the second tubular portion define a second cavity.

4. The syringe of claim 3 wherein the outlet valve assembly includes a third tubular portion with outlet opening and an activation opening, and disposed intermediate the second tubular portion and the chamber in a cruciform arrangement, and in communication therewith;
the third tubular portion for receiving an outlet valve member to operate the outlet opening between a closed position and an open position.

5. The syringe of claim 4 wherein the outlet valve member comprises:
a stem including a valve head adjacent to the activation opening, a valve base adjacent to the outlet opening, and a sleeve extending between valve head and the valve base;
the sealing member having:
a first annular ring extending from the sleeve, the first annular ring engaging the third tubular portion walls to seal the third tubular portion adjacent to the activation opening; and
a second annular ring extending from the sleeve, the second annular ring engaging the walls of the third tubular portion walls to seal the third tubular portion adjacent to the outlet opening;
the anchoring member having a plurality of lugs for engaging an annular recess in the walls of the third tubular portion to anchor the outlet valve member to maintain the outlet opening in the closed position;
wherein with the outlet opening in the closed position, the inlet valve member is driven towards the second tubular portion and the resilient member is compressed to open the inlet opening to allow fluid to flow via the first cavity and the second cavity into the chamber; and
wherein with the inlet opening in a closed position, the lugs are forced out of the annular recess such that a second annular ring completely disengages the walls of the second tubular portion to unseal the outlet opening and allow discharge from the chamber via the third tubular portion.

6. The syringe of claim 5 wherein the sleeve includes a plurality of longitudinally extending ribs which slidably engage the walls of the third tubular portion.

7. The syringe of claim 6 wherein the ribs are disposed between the first annular ring and the second annular ring, and the rings are equally spaced apart around the sleeve to define a plurality of third cavities between same, whereby the outlet valve member is dimensioned to occupy a large volume of the third tubular portion while allowing sufficient clearance between the walls of the third tubular portion for fluid flow via the third cavities without substantial impedance while preventing diminishing substantial fluid accumulation within the third tubular portion.

8. The syringe of claim 7 wherein the lugs are dimensioned such that they can not be forced back into the third outlet portion once the lugs have been forced out of the annular recess and the second annular ring completely disengages the walls of the second tubular.

9. The syringe of claim 8 wherein the first annular ring, the second annular ring, the ribs, and the lugs are formed integrally with the sleeve.

10. The syringe of claim 9 comprising a circuit for transmitting and receiving data related to the syringe or a patient, such as identification data, SKU, serial no., manufacturing date, expiry date, fluid data, patient data, health facility data, health practitioner data, medication data.

11. The syringe of claim 10 wherein the circuit for transmitting and receiving data comprises a transmitter, a receiver, a processor, a memory, an antenna and a power source.

12. The syringe of claim 11 wherein the circuit for transmitting and receiving data comprises radio identification (RFID) integrated circuit coupled to an antenna, wherein the RFID integrated circuit is passive, semi-passive, semi-active or active.

13. The syringe of claim 12 including at least one input/output device.

14. A syringe having:
a housing with a proximal end and a distal end;
an inlet valve assembly adjacent to the proximal end;
an outlet valve assembly adjacent to the proximal end;
the inlet valve assembly and the outlet valve assembly being angled with respect to one another;
the distal end of the housing having a chamber therein; the inlet valve assembly having:
an inlet opening, a first tubular portion leading to which the inlet opening leads, and a second tubular portion;
an inlet valve member received by the first tubular portion and the second tubular portion, to operate the inlet opening between an open position and a closed position;
the inlet valve member being dimensioned to occupy a substantial volume of the first tubular portion and the second tubular portion while defining a first cavity between the inlet valve member and the walls of the first tubular portion and defining a second cavity between the inlet valve member and the walls of the second tubular portion; and
a resilient member for biasing the inlet valve member in a closed position;
the outlet valve assembly having:
a third tubular portion with an activation opening and an outlet opening, and disposed intermediate the second tubular portion and the chamber in a cruciform arrangement, and in communication therewith;
the third tubular portion for receiving an outlet valve member to operate the outlet opening between a closed position and an open position;
the outlet valve member being dimensioned to occupy a substantial volume of the third tubular portion while defining a third cavity between the outlet valve member and the walls of the third tubular portion;
the outlet valve member having:
a first annular ring engaging the walls of the third tubular portion to seal the third tubular portion adjacent to the activation opening; and
a second annular ring engaging the walls of the third tubular portion to seal the third tubular portion adjacent to the outlet opening;
a plurality of lugs for engaging an annular recess in the walls of the third tubular portion to anchor the outlet valve member to maintain the outlet opening in the closed position;
wherein with the outlet opening in the closed position, the inlet valve member is driven towards the second tubular portion and the resilient member is compressed to open the inlet opening to allow fluid to flow via the first cavity and the second cavity into the chamber; and
wherein with the inlet opening in a closed position, the lugs are forced out of the annular recess such that a second annular ring completely disengages the walls of the second tubular portion to unseal the outlet opening and allow discharge from the chamber via the third cavity.
